(19) United States
(12) Patent Application Publication
(10) Pub. No.: US 2010/0330552 A1
(43) Pub. Date: Dec. 30, 2010

(54) Comparative cellular deficiency diagnosis genetic deviation diagnosis procedure cellular protein output deficiency

(76) Inventor: Curtis Murray, Draper, UT (US)

Correspondence Address: Curtis Murray, 1077 E. Hackamore Dr., Draper, UT, 84020, US

(21) Appl. No.: 12/455,199

(22) Filed: Jun. 30, 2009

(51) Int. Cl. C12Q 1/68; C12Q 1/02

(52) U.S. Cl. 435/6; 435/29

(57) ABSTRACT

This procedure is intended to demonstrate the genetic foundation of infirmities caused by abnormal base pair sequences and protein deficiencies (which are an expression of genetic aberrations).

Comparisons of normal and abnormal genetic sequences and protein outputs can be used to determine the root origin of the problem.

### CIRCUMSTANCES OF CONCEPTION

I have been studying genetics as a hobby for some time. I have come to realize that a easy accurate method for diagnosing genetic anomalies doesn't seem to be in practice or even available. After much consideration, I have deduced these procedures, they wouldn't have been possible until recently, and they can be useful.

### PURPOSE AND ADVANTAGES

The purpose of these procedures is to diagnose the exact genetic base pair code and protein deficiency errors of genetically caused infirmities. These diagnoses of the base pair code will allow others to focus on determining the solutions, treatments, and corrections to these genetically, caused disorders. By identifying the protein deficiency, the proteins can be provided by the process of re-combinant DNA procedures.

The advantage to these procedures is that they are relatively easy, quick, and inexpensive. It will be possible to ascertain the precise root of the problem. The dna and protein chain where these aberrant genes reside can readily be established today where it couldn't just a short time ago.

### SUMMARY OF IDEA

The idea is to compare dna and cellular protein output from healthy individuals with individuals inflicted with a genetically caused disorder. These comparisons will demonstrate the exact site of healthy deviation.

### DETAILED DESCRIPTION

Genetic Deviation Diagnosis Procedure

Non-Homo-Genetic Deviation
Definition—only cells within a specific area of the subject patient contain the genetic anomaly.
Example—a victim of cancer contain cells which are normal, but, cells in the cancer area contain a genetic anomaly which produces the disease.
Procedure—do a complete genetic base pair mapping of a cancer cell and a normal cell. A computer comparison will quickly distinguish the genetic difference which will be the exact site of the genetic deviation. Target proteins can then be sought for treatment.

Homo-Genetic Deviation
Definition—all of the cells of the patient contain the genetic anomaly.
Example—in a patient suffering from multiple sclerosis, all of the cell in the patient posses the same genetic anomaly.
Procedure;
   Do a complete genetic mapping of an adequately large community of people (possible 50) with the genetic disease.
   Do a complete genetic mapping of an equal sized community of people without the genetic disease.
   Compare the 2 groups;
      Determine at which genetic location all subjects are genetic homologous within their group, but, different between the two groups.
      Differing characteristics will be excluded if the subjects are chosen to represent an adequate cross section of types of people.
      Normal genetic variations will be ruled out because;
         Some traits will not be consistent within the group.
         Other traits will be similar in the different group.

Cellular Output Deficiency Diagnosis
Definition—a genetically caused disease in which cells of a hormone or protein producing organ fail to produce their appropriate chemical compounds.
Example—in a patient suffering from multiple sclerosis, the pituitary gland doesn't produce the requisite amounts of a protein necessary to control the muscles.
Procedure—culture a cellular sample from the appropriate (in this case pituitary) gland of a healthy and diseased subject or group.
Compare protein output—the sample from the genetic deviant individual or group will not contain the crucial protein or hormone which will be produced by the healthy individual or group
These proteins or hormones can be easily produced by re-combinant dna techniques.

### Ramifications, Scope, & Conclusions

These processes can be used to diagnose many diseases that have plagued man kind throughout his history. The first step for any treatment and cure is a complete and accurate diagnosis, which is what these procedures are designed to afford.

Though these procedures seem obvious and perhaps simple, they don't seem to be in practice throughout the scientific community. They can help in the search for ways to improve the quality of life for a great many people.

1. This process, that I've developed, though seemingly relative simple, is one that does not appear to be in use. By following this process, researchers can target the specific causes of genetically caused infirmities.
Improper DNA base pair sequence sections can be determined by comparison with proper ones.
Absent or improper protein output can be discovered by comparison of normal gland protein secretion with those of abnormal or absent secretions.
